(19) United States
(12) Patent Application Publication
(10) Pub. No.: US 2015/0297901 A1
(43) Pub. Date: Oct. 22, 2015

(54) APPARATUS AND METHOD RELATING TO AN ELECTROSTIMULATION DEVICE

(71) Applicant: KONINKLIJKE PHILIPS N.V., Eindhoven (NL)

(72) Inventor: FRANCISCUS NICOLAAS KOCKX, TEGELEN (NL)

(21) Appl. No.: 14/440,900

(22) PCT Filed: Nov. 21, 2013
(86) PCT No.: PCT/IB2013/060293

§ 371 Date: May 6, 2015

Related U.S. Application Data
(60) Provisional application No. 61/734,401, filed on Dec. 7, 2012.

(51) Int. Cl. A61N 1/372; A61N 1/36; A61N 1/04

(52) U.S. Cl. CPC A61N 1/37258; A61N 1/0456; A61N 1/36014

(57) ABSTRACT

An electrostimulation device for stimulating a user with one or more stimulation patterns via one or more electrodes, comprises a pattern generator adapted to generate one or more stimulation patterns for stimulating a user, and a detecting unit adapted to detect an operating state of the device. The pattern generator is further adapted to generate a notification signal in the form of a predetermined stimulation pattern, the notification signal corresponding to a detected operating state of the device.

### TECHNICAL FIELD

The present invention relates to an apparatus and method relating to an electrostimulation device, for example a Transcutaneous Electrical Nerve Stimulation (TENS) device.

### BACKGROUND

Electrostimulation devices such as Transcutaneous Electrical Nerve Stimulation devices, commonly known as TENS devices, stimulate a user using electrodes. The electrodes are typically connected to a TENS device either via a direct on-top connection or via lead wires. New TENS products are being targeted more at mobile usage, for example usage while the user is on the move, both inside and outside of the home. Portability and improved mobility is usually one of the attractions of these new devices.

FIG. 1 shows a typical TENS device 100, and comprises a display 101 for interfacing with a user, and control buttons 103 enabling a user to program the device. A pair of electrodes 105 are typically attached during use to the body of a user, and are used to provide electrical stimulation to the body of the user. FIG. 2 shows a typical pulsed signal, which is one of many pulsed signals that can be used to stimulate the body of the user. A speaker 107 is provided for outputting audible warning signals to the user, for example a low battery warning. A LED 107 may also be provided for outputting visual warnings to the user, for example that the device is in operation, or that the battery level is becoming low. Visual notifications may also be provided to the user via the display 101.

A disadvantage of such devices is that audible and visual indications given by the device tend to draw attention to the fact that the user is wearing such a device, thereby drawing attention to the fact that the user may be ill. This can discourage a user from benefiting from the mobility of such devices, since they are deterred from using the device in public because of the attention drawn to the device by the audiovisual warnings given by the device, for example given by the display 101, speaker 107 or LED 109.

One method for avoiding this problem would be to disable any audiovisual indicators given to the user. However, this has the disadvantage of requiring the user to perform regular check-ups on the device, for example by moving to a discrete location to check the current operating status of the device.

One example of such regular check-ups is to determine whether the battery is becoming low, since action is required before the battery is completely drained, otherwise under certain chronic conditions a drained battery can lead to some very unpleasant consequences for the user, for example because pain relief ceases to be available.

### SUMMARY

It is an aim of the present invention to provide a method and apparatus which obviate or reduce at least one or more of the disadvantages mentioned above.

According to a first aspect of the present invention there is provided an electrostimulation device for stimulating a user with one or more stimulation patterns via at least one or more electrodes. The device comprises a pattern generator adapted to generate one or more stimulation patterns for stimulating a user. A detecting unit is adapted to detect an operating state of the device. The pattern generator is further adapted to generate a notification signal in the form of a predetermined stimulation pattern, the notification signal corresponding to a detected operating state of the device.

The electrostimulation device is therefore adapted to provide feedback signals to the user via one or more electrodes normally used to provide stimulation therapy to the user, for example, thereby allowing the normal audio and/or visual signals to be disabled. This has the advantage that the device is less noticeable to third parties, for example when the device is used in a mobility mode of operation.

According to another aspect of the invention, there is provided a method in an electrostimulation device for providing a notification signal to a user relating to an operating state of the electrostimulation device, wherein the electrostimulation device comprises a pattern generator for generating one or more stimulation patterns for stimulating the user via one or more electrodes. The method comprises the steps of detecting an operating state of the device, and stimulating the user using a predetermined stimulation pattern, the predetermined stimulation pattern providing a notification signal to the user corresponding to the detected operating state of the device.

According to another aspect of the invention, there is provided a method in an electrostimulation device for improving the mobility of a user. The method comprises the steps of preventing any audio and/or visual notifications being given to a user of the device during a mobility mode of operation, and instead providing feedback to the user about an operating state of the electrostimulation device using one or more electrodes of the electrostimulation device.

### BRIEF DESCRIPTION OF THE DRAWINGS

For a better understanding of the present invention, and to show more clearly how it may be carried into effect, reference will now be made, by way of example only, to the following drawings in which:

FIG. 1 shows a typical electrostimulation device;

FIG. 2 shows a typical stimulation pattern for stimulating a user;

FIG. 3 shows an electrostimulation device according to an embodiment of the invention;

FIGS. 4a to 4e show examples of stimulation patterns that can be used to provide notification signals to a user;

FIG. 5 shows the steps performed by a method according to an embodiment of the present invention; and

FIG. 6 shows the steps performed by another embodiment.

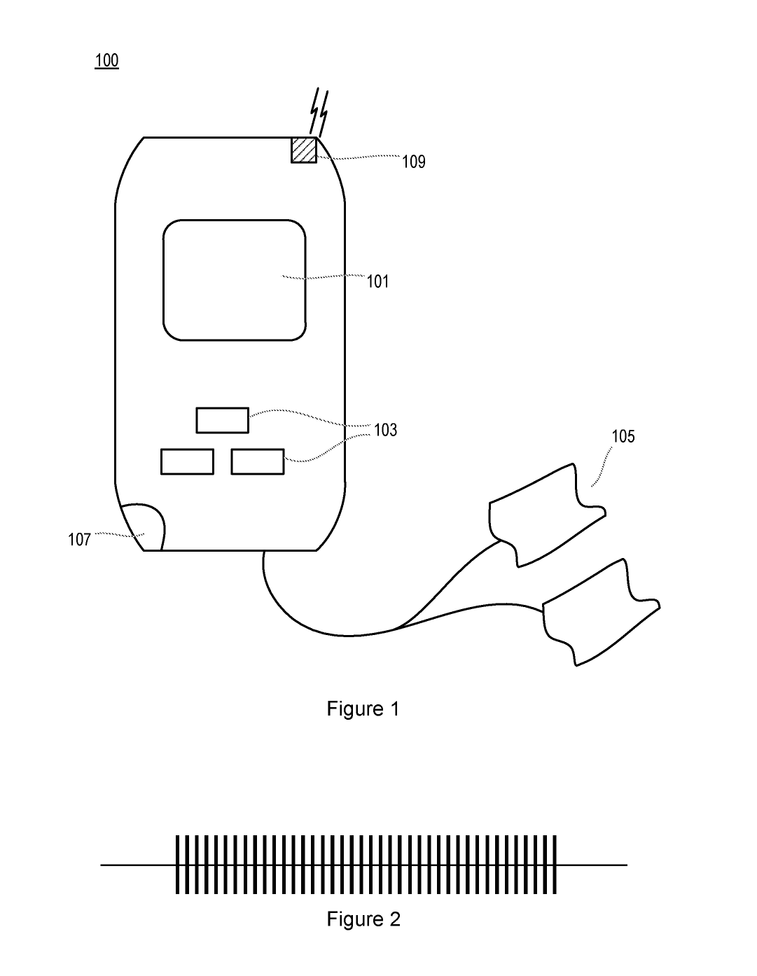

### DETAILED DESCRIPTION

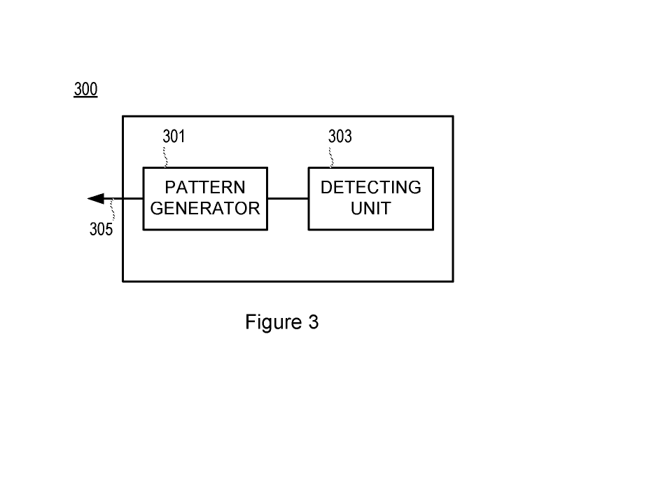

FIG. 3 shows an electrostimulation device 300 according to an embodiment of the invention, the electrostimulation device 300 being of the type suitable for stimulating a user with one or more stimulation patterns via one or more electrodes (not shown), for example to provide therapy or treatment to a user, or to provide comfort or relaxation to the user. The electrostimulation device 300 comprises a pattern generator 301 adapted to generate one or more stimulation patterns for stimulating a user. A detecting unit 303 is adapted to detect an operating state of the electrostimulation device. The pattern generator 301 is further adapted to generate a notification signal 305 in the form of a predetermined stimulation pattern, the notification signal 305 corresponding to a detected operating state of the device.

The notification signal 305 can be used to stimulate the user, during use, via one or more electrodes that are used in conjunction with the electrostimulation device for stimulating the user. For example, an embodiment of the invention can be used with one or more electrodes which are normally provided for treating the user. It is noted that the electrodes used to stimulate the user may comprise an electrode pair having an anode and a cathode, either formed as separate electrodes or integrated within one electrode or one body, or several such electrode pairs. As an alternative to using the actual electrodes that are used to provide therapy, one or more additional electrodes may be provided for the notification signal 305, such that the notification signal 305 can be used to provide a notification signal to a user alongside the electrodes that are used to provide therapy to the user. In such a system a selecting circuit may be provided to enable a selection to be made regarding which electrodes should be used to provide the notification signal. Furthermore, in an embodiment having two or more pairs of electrodes, the system may be configured such that the two or more pairs of electrodes provide therapy during normal operation, with one pair of electrodes being removed from normal operation during a period when a notification signal needs to be notified to the user, with that pair of electrodes then being used to provide the notification signal during such periods.

By providing feedback signals to the user via one or more electrodes, this means that audio and/or visual signals can be disabled, such that the device is less noticeable to third parties, for example when the device is used in a mobility mode.

It is noted that the detecting unit 303 may comprise a monitoring device for monitoring a parameter or characteristic of the electrostimulation device, such as monitoring a charge level of a battery to detect a low battery level.

In some embodiments the electrostimulation device may be formed within a single housing, for example whereby the pattern generator and user interface devices are located within the same housing.

In other embodiments, however, the electrostimulation device may comprise a main body, for example a control unit that comprises a pattern generator (to which the electrodes are connected), and a remote unit that comprises user interface means for enabling a user to control the electrostimulation device as a whole. In such an embodiment, the detecting unit 303 of FIG. 3 may comprise a receiver device for receiving a warning signal that is being communicated from the remote device, for example warning that a battery of the remote control device is becoming low. Upon the detecting unit 303 receiving this warning signal, the pattern generator 301 is then able to provide an appropriate notification signal 305 to the user. As such, it will be appreciated that the detecting unit 303 is intended to embrace any circuit or device for determining an operating state of the electrostimulation device, whether the operating state of the device be part of the same physical housing as the pattern generator, or part of a remote device which works in association with a main device which houses the pattern generator.

The pattern generator 301 can be adapted to generate a plurality of notification signals, each notification signal having a corresponding stimulation pattern, and each notification signal notifying a particular operating state of the device.

The embodiments of the invention are intended to embrace any operating state of the electrostimulation device being detected and notified to the user, including but not limited to one or more of: a charge level of a power source; a charge level of a power source being below a threshold level; a status of a timer module; a status flag relating to a remote control device associated with the electrostimulation device being outside a predetermined range. It is noted that a charge level can relate to a charge level of a single unitary device forming the electrostimulation device, or a charge level of a main device and/or auxiliary remote device which in combination form the electrostimulation device. The operating state of the device can also relate to a warning state.

A stimulation pattern may comprise a predefined characteristic for notifying a particular operating state of the device to the user. The predetermined characteristic may be a frequency of a pulsed signal, an amplitude of a pulsed signal, or a combination of both.

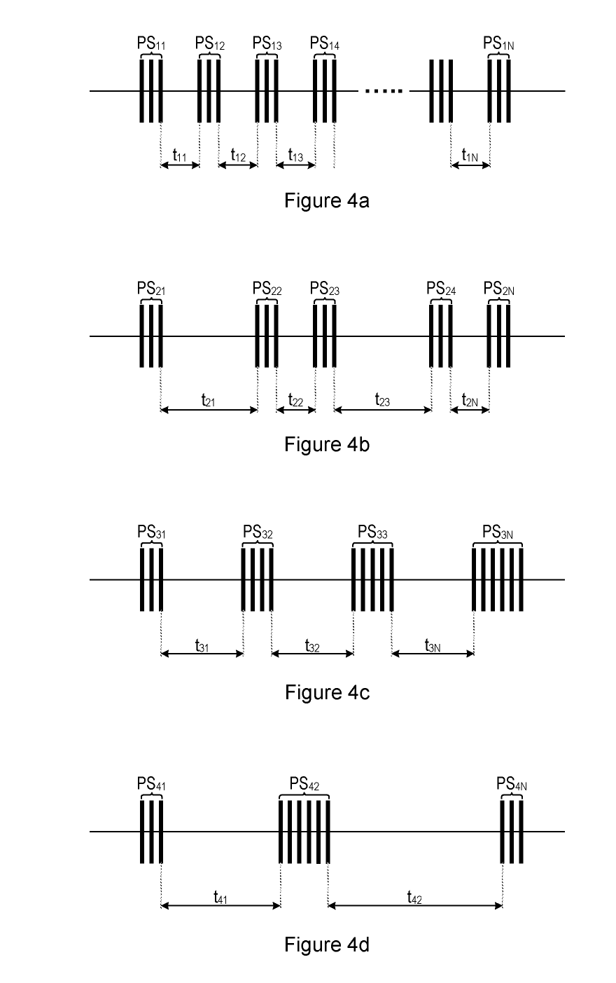

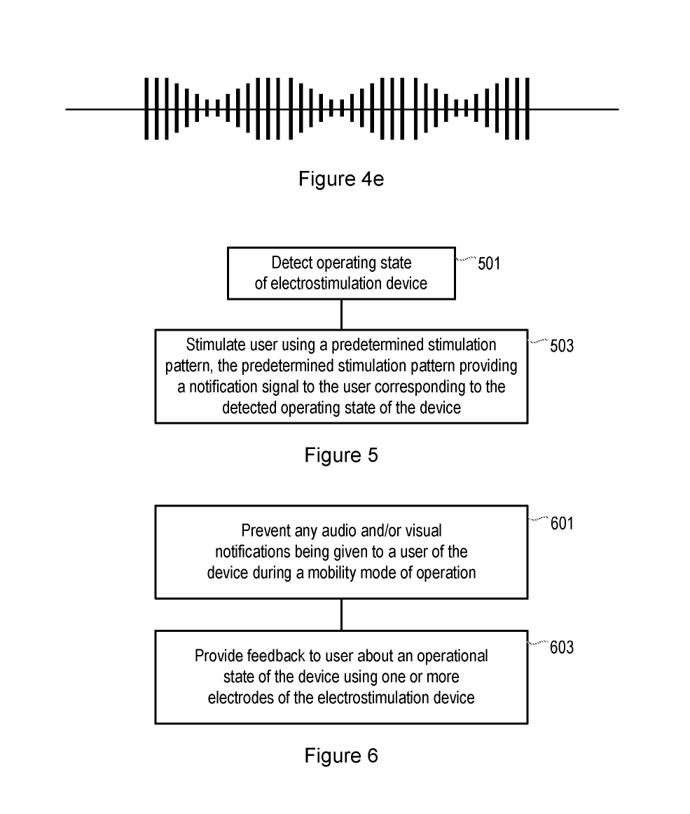

Examples of notification signals and their corresponding stimulation patterns are provided in FIGS. 4a to 4e. It is noted that, although the pulses shown in FIGS. 4a to 4e are bi-phasic in nature, the embodiments of the invention are intended to cover all forms of signals that can be used to provide a notification signal to a user via the electrodes, including mono phase pulsed signals, symmetrical or asymmetrical pulsed signals.

According to one embodiment a notification signal comprises a pulse series as shown in FIG. 4a, comprising a series of pulses of equal amplitude PS₁₁ to PS_{1N}, wherein the pulse series comprises one or more pause intervals t₁₁ to t_{1N}. For example, the notification signal may comprise a pulse series comprising a series of pulses of equal amplitude PS₁₁ to PS_{1N}, for example having equal burst durations of 3 pulses in the example of FIG. 4a, and wherein the pulse series comprises one or more pause intervals t₁₁ to t_{1N}. In the example of FIG. 4a the pause intervals t₁₁ to t_{1N} are shown as having equal duration. It is noted, however, that different pause intervals may be used, for example pause intervals that become gradually longer, pause intervals that become gradually shorter, or long pause intervals being interleaved with short pause intervals.

FIG. 4b shows an example whereby a notification signal comprises a pulse series comprising a series of pulses of equal amplitude PS₂₁ to PS_{2N}, wherein the pulse series comprises one or more pause intervals t₂₁ to t_{2N}, and wherein the duration of a pause interval t₂₁ to t₂ₙ is different to at least one other pause interval t₂₁ to t_{2N}.

It can be seen that the notification signals shown in FIGS. 4a and 4b are examples of a pulse series comprising a series of pulses of equal amplitude PS₁₁ to PS_{1N}; PS₂₁ to PS_{2N}, wherein the pulse series comprises one or more pause intervals t₁₁ to t_{1N}; t₂₁ to t_{2N}, and wherein the number of pulses PS₁₁ to PS_{1N}; PS₂₁ to PS_{2N} between each pause interval is equal, i.e. 3 pulses in the examples. It is noted that any number of pulses can be provided in such embodiments.

According to another embodiment, the notification signal comprises a pulse series comprising a series of pulses of equal amplitude PS₃₁ to PS_{3N}; PS₄₁ to PS_{4N}, as shown in FIGS. 4c and 4d, wherein the pulse series comprises one or more pause intervals t₃₁ to t_{3nN}, t₄₁ to t_{4N}, and wherein the number of pulses between a first pause interval and a second pause interval is different to the number of pulses between a third pause interval and a fourth pause interval.

For example, FIG. 4c shows an example whereby the number of pulses increases after each pause interval, for example 3 pulses in PS₃₁, 4 pulses in PS₃₂, 5 pulses in PS₃₃, to N pulses in PS_{3N}. Alternatively, the number of pulses could be arranged to decrease. In these embodiments the pulse duration is effectively being changed. It is noted that although FIG. 4c shows pause intervals t₃₁ to t_{3N} of equal duration, the pause intervals may also be arranged in some pattern, if desired.

The example of FIG. 4d has pulse numbers which rise and fall between different pause intervals, and shows pause intervals for t₄₁ and t₄₂ having different durations.

It is noted that other stimulation patterns may be provided as predetermined notification signals, including in any combination of the formats described above, all of which are intended to fall within the scope of the appended claims. An electrostimulation device can be adapted to select a chosen set of stimulation patterns that correspond to a set of notification signals, such that the user can become familiar with that set of notification signals. Also, the system may be configurable such that the user can choose which stimulation patterns are associated with which notifications signals, such that the user can select which notification signal is received when a battery becomes low, for example.

According to another embodiment shown in FIG. 4e, a notification signal may comprises a pulse series, wherein the amplitudes of the pulses in the pulse series are modulated. In other words, a stimulation pattern corresponding to a notification signal comprises a pulse series that has been modulated with a modulation pattern. For example, the modulation pattern may comprise a sinusoidal pattern, or a linear ramp-up, or a liner ramp down, or some other noticeable function.

It will therefore be appreciated that the electrostimulation device 300 of FIG. 3 may be adapted such that the pattern generator 301 is configured to selectively generate one or more notification signals during a mobility mode of operation.

According to one embodiment, the electrostimulation device comprises a disable unit for disabling operation of audio and/or visual notifications during a mobility mode of operation.

FIG. 5 shows a method according to another embodiment of the invention. The method may be performed in an electrostimulation device for providing a notification signal to a user relating to an operating state of the electrostimulation device. The electrostimulation device may be of the type comprising a pattern generator for generating one or more stimulation patterns for stimulating the user via one or more electrodes. The method comprises the step detecting an operating state of the device, step 501. In step 503, a user is stimulated using a predetermined stimulation pattern, the predetermined stimulation pattern providing a notification signal to the user corresponding to the detected operating state of the device.

The method may comprise the steps of generating a plurality of notification signals, each notification signal corresponding to a particular operating state of the device, and stimulating the user with one of the notification signals in response to detecting a corresponding operating state of the electrostimulation device.

As mentioned above, an operating state of the device may comprises one or more of: a charge level of a power source; a charge level of a power source being below a threshold level; a status of a timer module; a status flag relating to a remote control device associated with the electrostimulation device being outside a predetermined range. Other operating states of the device may also be notified using a notification signal.

The step of stimulating the user may comprise the steps of stimulating the user with a pulse series comprising a series of pulses of equal amplitude PS₁₁ to PS_{1N}, wherein the pulse series comprises one or more pause intervals t₁₁ to t_{1N}. According to an alternative embodiment, the step of stimulating the user comprises the steps of stimulating the user with a pulse series comprising a series of pulses of equal amplitude PS₁₁ to PS_{1N}, wherein the pulse series comprises one or more pause intervals t₁₁ to t_{1N}, and wherein the pause intervals t₁₁ to t_{1N} have equal duration. According to an alternative embodiment, the step of stimulating the user comprises the steps of stimulating the user with a pulse series comprising a series of pulses of equal amplitude PS₂₁ to PS_{2N}, wherein the pulse series comprises one or more pause intervals t₂₁ to t_{2N}, and wherein the duration of a pause interval t₂₁ to t_{2N} is different to at least one other pause interval t₂₁ to t_{2N}. According to an alternative embodiment, the step of stimulating the user comprises the steps of stimulating the user with a pulse series comprising a series of pulses of equal amplitude PS₁₁ to PS_{1N}; PS₂₁ to PS_{2N}, wherein the pulse series comprises one or more pause intervals t₁₁ to t_{1N}; t₂₁ to t_{2N}, and wherein the number of pulses PS₁₁ to PS_{1N}; PS₂₁ to PS_{2N} between each pause interval is equal. According to an alternative embodiment, the step of stimulating the user comprises the steps of stimulating the user with a pulse series comprising a series of pulses of equal amplitude PS₃₁ to PS_{3N}; PS₄₁ to PS_{4N}, wherein the pulse series comprises one or more pause intervals t₃₁ to t_{3N}; t₄₁ to t_{4N}, and wherein the number of pulses between a first pause interval and a second pause interval is different to the number of pulses between a third pause interval and a fourth pause interval.

The method may further comprise the step of modulating the amplitude of a pulse series with a modulation pattern.

FIG. 6 shows the steps performed by a method according to another embodiment of the invention, for improving the mobility of a user of an electrostimulation device. The method comprises the steps of preventing any audio and/or visual notifications being given to a user of the device during a mobility mode of operation, step 601. Feedback is provided to the user about an operating state of the electrostimulation device, instead, using one or more electrodes of the electrostimulation device, step 603.

It can be seen from above that the embodiments of the invention are concerned with giving feedback to user via the electrodes of an electrostimulation device, for example the electrodes already provided for performing the electrostimulation. The feedback may be given in a number of ways, by pulsing or modulating the signal in a particular way which is distinguishable to the user from the stimulation signals used for normal therapy.

The pulse generator used to provide pulse trains or pulse patterns for electrostimulation therapy is adapted such that the pulse generator is controlled, based on some operating parameter or status of the device changing, to provide an indicator to the user via the electrodes.

A set of special pulse trains or patterns can therefore be provided, for example as shown in FIGS. 4a to 4e, different to the pulse trains or patterns provided for pain relief, that are based or triggered or related to a particular operating state or status of the device.

As a consequence, this has the advantage of avoiding any unnecessary attention being drawn to the TENS device by any audiovisual user interface, which is especially unwanted in public places.

As TENS is an active treatment a difference in an output can be clearly identified by the user. In other words, if a user is receiving stimulation based on a repetitive stimulation pattern, then an interruption or change in that pattern, corresponding to a notification signal, caused by a change in the operating status of the device, will be clearly noticeable to the user.

In one embodiment, the invention may comprise pausing a TENS pulse according to a predetermined pattern, for example where the TENS pulse is the pulse train or pattern already being used to provide therapy. With such an embodiment, the normal therapy being experienced by the user will contain noticeable pauses or patterns to provide a notification signal corresponding to a certain operating state of the device to the user. In other words, an existing stimulation pattern is mixed or adapted to provide a notification signal therein. For example, a series of long pulses can be provided within the existing stimulation pattern being applied, which can be used to show that the battery level is becoming low.

The user will notice these differences in output due to the “TENS” feeling the user experiences from the device. The patterns (which can take many forms) can be set to last for different durations. For example, the notification signals may be applied continuously until the user takes some form of action (such as replacing the battery). Alternatively, the notification signals can be applied for a limited period, thereby giving the user notification for a set period, and then continuing with the normal stimulation signal used for therapy. In this way the notification signal is provided intermittently with the stimulation signal that is used for therapy.

The embodiments of the invention may be retro fitted to existing electrostimulation devices such as TENS devices, by programming the pattern generators to provide these additional forms of notification signals. User interface means may be provided, or adapted, to enable the user to selectively operate the non-audible non-visual notification signals. For example, a “mobility mode” (or “silent mode”) may be provided, such that the user can easily place the device in the mobility mode.

The embodiments of the invention have the advantage that they help disguise that a user is wearing a TENS device, and therefore have the advantage of not drawing the attention of other people to the fact that a device is being worn by the user, and that the user may be ill.

The embodiments of the invention also have the advantage of providing notification or indicator signals to a user in a discrete way. Discrete means that the device is not visible and not drawing attention, and does not require regular check-ups of the controller of the device to determine the current device status.

Because user interfaces with LEDs and/or audible feedback draw attention from others beside the user itself, the embodiments of the invention provide a way to give feedback without other people noticing. The user will in this case be able to go to a discrete location to check the device.

It is noted that the embodiments of the invention described above may be used with any type of electrostimulation device, or retrofitted to any type of electrostimulation device. In other words, the embodiments of the invention may be used with electrostimulation devices having any arrangement of display device (or devices that do not have a display device, or where the display device is provided in a remote unit), or any arrangement of input interface, or any arrangement for providing audible warnings. The embodiments may also be used with single devices contained within the same housing, or mainauxiliary type devices, whereby the function of the electrostimulation device is split between a main device and an auxiliary device.

It is also noted that the term “operating state” can be used in some embodiments to indicate a warning state of the electrostimulation device, or a warning mode of operation.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single processor or other unit may fulfil the functions of several units recited in the claims. Any reference signs in the claims shall not be construed so as to limit their scope.

1. An electrostimulation device for stimulating a user with one or more stimulation patterns via one or more electrodes, the device comprising:
a pattern generator adapted to generate one or more stimulation patterns for stimulating the user;
a detecting unit adapted to detect an operating state of the device;
wherein the pattern generator is further adapted to generate a notification signal in the form of a predetermined stimulation pattern, the notification signal corresponding to a detected operating state of the device;
wherein the pattern generator is configured to selectively generate one or more notification signals during a mobility mode of operation.

2. An electrostimulation device as claimed in claim 1, wherein the pattern generator is adapted to generate a plurality of notification signals, each notification signal having a corresponding stimulation pattern, and each notification signal notifying a particular operating state of the device.

3. An electrostimulation device as claimed in claim 1, wherein an operating state of the device comprises one or more of:
a charge level of a power source;
a charge level of a power source being below a threshold level;
a status of a timer module;
a status flag relating to a remote control device associated with the electrostimulation device being outside a predetermined range.

4. An electrostimulation device as claimed in claim 1, wherein a stimulation pattern comprises a predefined characteristic for notifying a particular operating state of the device to the user.

5. An electrostimulation device as claimed in claim 1, wherein a stimulation pattern corresponding to a notification signal comprises one or more of:
a pulse series comprising a series of pulses of equal amplitude (PS₁₁ to PS_{1N}), wherein the pulse series comprises one or more pause intervals (t₁₁ to t_{1N});
a pulse series comprising a series of pulses of equal amplitude (PS₁₁ to PS_{1N}), wherein the pulse series comprises one or more pause intervals (t₁₁ to t_{1N}), and wherein the pause intervals (t₁₁ to t_{1N}) have equal duration;
a pulse series comprising a series of pulses of equal amplitude (PS₂₁ to PS_{2N}), wherein the pulse series comprises one or more pause intervals (t₂₁ to t_{2N}), and wherein the duration of a pause interval (t₂₁ to t_{2N}) is different to at least one other pause interval (t₂₁ to t_{2N});
a pulse series comprising a series of pulses of equal amplitude (PS₁₁ to PS_{1N}; PS₂₁ to PS_{2N}), wherein the pulse series comprises one or more pause intervals (t₁₁ to t_{1N}; t₂₁ to t_{2N}), and wherein the number of pulses (PS₁₁ to PS_{1N}; PS₂₁ to PS_{2N}) between each pause interval is equal;
a pulse series comprising a series of pulses of equal amplitude (PS₃₁ to PS_{3N}; PS₄₁ to PS_{4N}), wherein the pulse series comprises one or more pause intervals (t₃₁ to t_{3N}; t₄₁ to t_{4N}), and wherein the number of pulses between a first pause interval and a second pause interval is different to the number of pulses between a third pause interval and a fourth pause interval.

6. An electrostimulation device as claimed in claim 5, wherein a stimulation pattern corresponding to a notification signal further comprises a pulse series that has been modulated with a modulation pattern.

7. An electrostimulation device as claimed in claim 1, wherein a stimulation pattern corresponding to a notification signal comprises a pulse series that has been modulated with a modulation pattern.

8. (canceled)

9. An electrostimulation device as claimed in claim 1, further comprising a disable unit for disabling operation of audio and/or visual notifications during the mobility mode of operation.

10. A method in an electrostimulation device for providing a notification signal to a user using an electrostimulation device, wherein the electrostimulation device comprises a pattern generator for generating one or more stimulation patterns for stimulating the user via one or more electrodes, the method comprising the steps of:
detecting an operating state of the device; and
stimulating the user using a predetermined stimulation pattern, the predetermined stimulation pattern providing a notification signal to the user corresponding to the detected operating state of the device.

11. A method as claimed in claim 10, further comprising the steps of generating a plurality of notification signals, each notification signal corresponding to a particular operating state of the device, and stimulating the user with one of the notification signals in response to detecting a corresponding operating state of the electrostimulation device.

12. A method as claimed in claim 10 wherein an operating state of the device comprises one or more of:
a charge level of a power source;
a charge level of a power source being below a threshold level;
a status of a timer module;
a status flag relating to a remote control device associated with the electrostimulation device being outside a predetermined range.

13. A method as claimed in claim 10, wherein the step of stimulating the user comprises the step of:
stimulating the user with a pulse series comprising a series of pulses of equal amplitude (PS₁₁ to PS_{1N}), wherein the pulse series comprises one or more pause intervals (t₁₁ to t_{1N}); or
stimulating the user with a pulse series comprising a series of pulses of equal amplitude (PS₁₁ to PS_{1N}), wherein the pulse series comprises one or more pause intervals (t₁₁ to t_{1N}), and wherein the pause intervals (t₁₁ to t_{1N}) have equal duration; or
stimulating the user with a pulse series comprising a series of pulses of equal amplitude (PS₂₁ to PS_{2N}), wherein the pulse series comprises one or more pause intervals (t₂₁ to t_{2N}), and wherein the duration of a pause interval (t₂₁ to t_{2N}) is different to at least one other pause interval (t₂₁ to t_{2N}); or
stimulating the user with a pulse series comprising a series of pulses of equal amplitude (PS₁₁ to PS_{1N}; PS₂₁ to PS_{2N}), wherein the pulse series comprises one or more pause intervals (t₁₁ to t_{1N}; t₂₁ to t_{2N}), and wherein the number of pulses (PS₁₁ to PS_{1N}; PS₂₁ to PS_{2N}) between each pause interval is equal; or
stimulating the user with a pulse series comprising a series of pulses of equal amplitude (PS₃₁ to PS_{3N}; PS₄₁ to PS_{4N}), wherein the pulse series comprises one or more pause intervals (t₃₁ to t₃ₙ; t₄₁ to t₄ₙ), and wherein the number of pulses between a first pause interval and a second pause interval is different to the number of pulses between a third pause interval and a fourth pause interval.

14. A method as claimed in claim 10, wherein the step of stimulating the user comprises the step of modulating the amplitude of a pulse series with a modulation pattern.

15. A method in an electrostimulation device for improving the mobility of a user, the method comprising the steps of preventing any audio and/or visual notifications being given to a user of the device during a mobility mode of operation, and instead providing feedback to the user about an operating state of the electrostimulation device using one or more electrodes of the electrostimulation device.
